(19) United States
(12) Patent Application Publication
(10) Pub. No.: US 2026/0083354 A1
(43) Pub. Date: Mar. 26, 2026

(54) SYSTEM AND A METHOD FOR MONITORING MUSCLE ACTIVITIES AND 5 PROVIDING FEEDBACK THEREOF

(71) Applicant: Yogesh Navaneethan, London (GB)

(72) Inventors: Yogesh Navaneethan, London (GB); Vaishnavi Sundararajan, Chennai (IN)

(73) Assignee: Yogesh Navaneethan, London (GB)

(21) Appl. No.: 19/309,851

(22) Filed: Aug. 26, 2025

(30) Foreign Application Priority Data
Sep. 24, 2024 (IN) 202411072010

(51) Int. Cl. A61B 5/11; A61B 5/00; A61B 5/397; G16H 10/60; G16H 40/67

(52) U.S. Cl. CPC A61B 5/1114; A61B 5/0004; A61B 5/397; A61B 5/7264; G16H 10/60; G16H 40/67; A61B 2560/0209; A61B 2562/04

(57) ABSTRACT

The present invention relates to a system and method to monitor and provide real-time feedback on core and lower back muscle engagement during physical activities. Further, the system is designed to enhance physical performance, and support rehabilitation by utilizing advanced biosensors, AI-driven data analysis, and customizable feedback mechanisms. The method involves continuously analyzing electromyographic signals from the targeted muscle groups. As such, the present invention ensures that users receive actionable insights tailored to their specific needs, making the system a powerful tool for fitness, health, and rehabilitation applications. The feedback is provided to the user through a user device including a mobile phone, a Personal Computer (PC), and a datalogger gadget. The user device is connected to the system using a wired or wireless medium.

### TECHNICAL FIELD

The present disclosure relates to the field of medical devices. More particularly, it pertains to a system and method for monitoring muscle activities and providing feedback thereof.

### BACKGROUND

In the rapidly evolving landscape of fitness technology and rehabilitation, the need for precise and real-time monitoring of muscle engagement has become increasingly critical. Core and lower back muscles play a pivotal role in maintaining stability and enhancing performance. However, existing technologies often fail to provide targeted, accurate feedback for these crucial muscle groups, leading to suboptimal training outcomes. As individuals across various fitness levels seek more personalized and effective training tools, the demand for advanced systems that can deliver real-time, actionable insights into muscle engagement continues to grow.

Conventional methods available for monitoring muscle activity primarily include surface electromyography (sEMG) systems, intramuscular electromyography (iEMG) systems, and basic fitness trackers with limited EMG capabilities. sEMG systems, while non-invasive, often lack the specificity needed to accurately monitor deep core and lower back muscles. iEMG systems provide more precise data but are invasive, uncomfortable, and impractical for everyday use. Fitness trackers with EMG sensors typically offer only basic muscle activity data, which is insufficient for users who need detailed feedback during dynamic workouts. These traditional systems also commonly rely on post-activity data analysis, which does not allow for real-time adjustments, and often use visual or auditory feedback methods that can be distracting during exercise. Additionally, these systems do not adapt to individual users over time, limiting their effectiveness in providing personalized guidance.

Additionally, wearable fitness trackers with basic EMG capabilities have gained popularity for their convenience and ability to monitor general muscle activity. However, these devices are typically designed for broader fitness applications and lack the accuracy and depth of analysis required for targeted muscle engagement monitoring. Moreover, these systems usually provide feedback after the activity has been completed, making it difficult for users to make real-time adjustments to their form or muscle engagement. The reliance on visual or auditory feedback mechanisms in these conventional systems can also be distracting during physical activities, further limiting their effectiveness.

To address the technical problems associated with these conventional methods, there is a need for a system and method that can provide real-time, personalized feedback specifically focused on core and lower back muscle engagement. Thus, there is a need for a system and method to monitor and provide real-time feedback on core and lower back muscle engagement.

Further limitations and disadvantages of conventional and traditional approaches will become apparent to one of skill in the art, through the comparison of described systems with some aspects of the present disclosure, as set forth in the remainder of the present application and with reference to the drawings.

### SUMMARY

A system to monitor and provide real-time feedback on core and lower back muscle engagement during physical activities is disclosed. The system comprises a sensing unit comprising a plurality of biosensors physically coupled to a user. The plurality of biosensors is configured to detect electromyographic signals from the core and lower back muscles of the user. The system further comprises an operational unit communicatively coupled with a plurality of biosensors. The operation unit comprises a processor configured to obtain the electromyographic signals from the operational unit. The processor is further configured to analyze the obtained electromyographic signals in real time to determine the muscle activity of the user. The muscle activity is indicative of movement of core and/or lower back muscles. The processor is further configured to generate activity data based on the muscle activity of the user. The system further comprises an output unit communicatively coupled with the operational unit. The output unit is configured to generate feedback for the user, based on the activity data, and provide the feedback to the user through one or more of a haptic output, a visual output, and an auditory output.

In an aspect, the processor is further configured to securely store raw data and processed data in memory for future reference and analysis.

In an aspect, the processor is further configured to protect the integrity and confidentiality of the data processed, stored, and transmitted using an encryption method.

In an aspect, the processor is further configured to obtain the user's historical data from the memory, determine, using the Machine Learning (ML) model, individual muscle engagement patterns based on the user's historical data, and continuously update the feedback based on the individual muscle engagement patterns.

In an aspect, the processor is further configured to allow the user to customize the type, intensity, and mode of feedback delivery according to their preferences.

In an aspect, the system further comprises a communication network configured to facilitate the transmission of electromyographic data between the sensing unit, the operational unit, and the output unit.

In an aspect, the system further comprises a power management unit configured to manage power distribution, battery charging, and energy efficiency across all components of the system.

In an aspect, the feedback is provided to the user through a user device including a mobile phone, a Personal Computer (PC), and a datalogger gadget.

In an aspect, the user device is connected to the system using a wired or wireless medium.

In an aspect, the user device is equipped with an application for providing an external communication capability to the system.

In an aspect, the user device is configured to transmit the processed information to an external device or server that comprises an Artificial Intelligence (AI) capable processor for processing the activity data.

In an embodiment, a method for monitoring and providing real-time feedback on core and lower back muscle engagement is disclosed. The method comprises detecting electromyographic signals from core and lower back muscles using a sensing unit that comprises a plurality of biosensors. The method further comprises transmitting the electromyographic signals to an operation unit comprising a processor. The method further comprises analyzing, by the processor, the electromyographic signals in real-time to determine the muscle activity of the user. The muscle activity is indicative of movement of core and/or lower back muscles. The method further comprises generating, by the processor, activity data based on the muscle activity of the user. The activity data is provided to an output unit. The method further comprises generating, by the output unit, feedback for the user based on the activity data. The method further comprises providing, by the output unit, the feedback to the user through one or more of a haptic output, a visual output, and an auditory output.

### BRIEF DESCRIPTION OF DRAWINGS

The accompanying drawings illustrate the various embodiments of systems, methods, and other aspects of the disclosure. Any person with ordinary skills in the art will appreciate that the illustrated element boundaries (e.g., boxes, groups of boxes, or other shapes) in the figures represent one example of the boundaries. In some examples, one element may be designed as multiple elements, or multiple elements may be designed as one element. In some examples, an element shown as an internal component of one element may be implemented as an external component in another, and vice versa. Further, the elements may not be drawn to scale.

Various embodiments will hereinafter be described in accordance with the appended drawings, which are provided to illustrate and not to limit the scope in any manner, wherein similar designations denote similar elements, and in which:

FIG. 1 is a block diagram that illustrates a system environment in which various embodiments of the method and the system may be implemented.

FIG. 2 is a block diagram that illustrates an application server configured for the system and method to monitor electromyography (EMG) of lower back and core muscles, in accordance with an embodiment of the present disclosure.

FIG. 3 illustrates a system for monitoring and providing real-time feedback on muscle engagement, in accordance with an embodiment of the present disclosure.

FIG. 4 illustrates a block diagram of the operational unit, in accordance with an embodiment of the present disclosure.

FIG. 5 illustrates a block diagram of the output unit, in accordance with an embodiment of the present disclosure.

FIG. 6 illustrates a flowchart of a method for monitoring and providing real-time feedback on muscle engagement, in accordance with an embodiment of the present disclosure.

FIG. 7 is a flowchart that illustrates a method for monitoring electromyography (EMG) of the lower back and core muscles, in accordance with an embodiment of present disclosure.

FIG. 8 is a flow chart that illustrates how the processor is configured to monitor electromyography (EMG) of lower back and core muscles, in accordance with an embodiment of present disclosure.

### DETAILED DESCRIPTION

The present disclosure may be best understood with reference to the detailed figures and description set forth herein. Various embodiments are discussed below with reference to the figures. However, those skilled in the art will readily appreciate that the detailed descriptions given herein with respect to the figures are simply for explanatory purposes as the methods and systems may extend beyond the described embodiments. For example, the teachings presented and the needs of a particular application may yield multiple alternative and suitable approaches to implement the functionality of any detail described herein. Therefore, any approach may extend beyond the particular implementation choices in the following embodiments described and shown.

References to “one embodiment,” “at least one embodiment,” “an embodiment,” “one example,” “an example,” “for example,” and so on indicate that the embodiment(s) or example(s) may include a particular feature, structure, characteristic, property, element, or limitation but that not every embodiment or example necessarily includes that particular feature, structure, characteristic, property, element, or limitation. Further, repeated use of the phrase “in an embodiment” does not necessarily refer to the same embodiment.

The present disclosure addresses the limitations of conventional electromyography systems by introducing a novel system designed specifically for monitoring and providing real-time feedback on core and lower back muscle engagement. The system incorporates advanced biosensors that accurately detect electromyographic signals from these critical muscle groups, overcoming the generalization and imprecision of traditional methods. The system utilizes a processor to analyze the signals in real-time, allowing for immediate, personalized feedback through haptic, visual, and auditory outputs. The system continuously adapts to the user's unique muscle engagement patterns, providing tailored insights that optimize performance, and support effective rehabilitation. Additionally, the system integrates seamlessly with digital health platforms, offering comprehensive data logging, connectivity, and safety features that ensure reliable and user-friendly operation across various fitness and rehabilitation scenarios.

The primary objective of the present disclosure is to provide a system that offers accurate, real-time monitoring and feedback specifically for core and lower back muscle engagement during physical activities. To achieve this, the present disclosure aims to overcome the limitations of conventional electromyography systems by integrating advanced biosensors with processing circuitry, enabling precise detection and analysis of muscle activity. The system's objective is to deliver immediate, personalized feedback through haptic, visual, and auditory outputs, allowing users to make real-time adjustments that optimize performance. Additionally, the present disclosure seeks to create a user-friendly, non-invasive solution that adapts to individual user patterns over time. The system also aims to seamlessly integrate with digital health platforms, providing comprehensive data logging, connectivity, and safety features to support a wide range of fitness and medical applications.

The present invention is a system designed to monitor and provide real-time feedback on core and lower back muscle engagement during physical activities, addressing the shortcomings of conventional electromyography systems. The system features advanced biosensors specifically calibrated to detect the subtle electromyographic signals generated by these critical muscle groups. These signals are processed by a processor to analyze muscle activity in real-time, providing immediate, personalized feedback through haptic, visual, and auditory outputs. Its ability to adapt dynamically to the user's unique muscle engagement patterns, offering tailored insights that evolve with continued use, thereby optimizing performance, and supporting effective rehabilitation. Further, the system's seamless integration with digital health platforms, enabling comprehensive data logging, predictive analytics, and real-time connectivity, making it a versatile and user-friendly tool for both fitness and medical applications.

Further, the feedback is provided to the user through a user device including a mobile phone, a Personal Computer (PC), and a datalogger gadget. The user device is connected to the system using a wired or wireless medium. The user device is equipped with an application for providing an external communication capability to the system. The user device comprises an Artificial Intelligence (AI) capable processor for processing the activity data. The AI-capable processor is a special-purpose processing unit used in computer systems employed for artificial intelligence and machine learning tasks. The AI-capable processors are optimized to process data based on user's instructions as well as a self-learning/machine learning approach. AI-capable processors are different from traditional processors because they can perform many calculations in parallel, which can make them faster and more efficient.

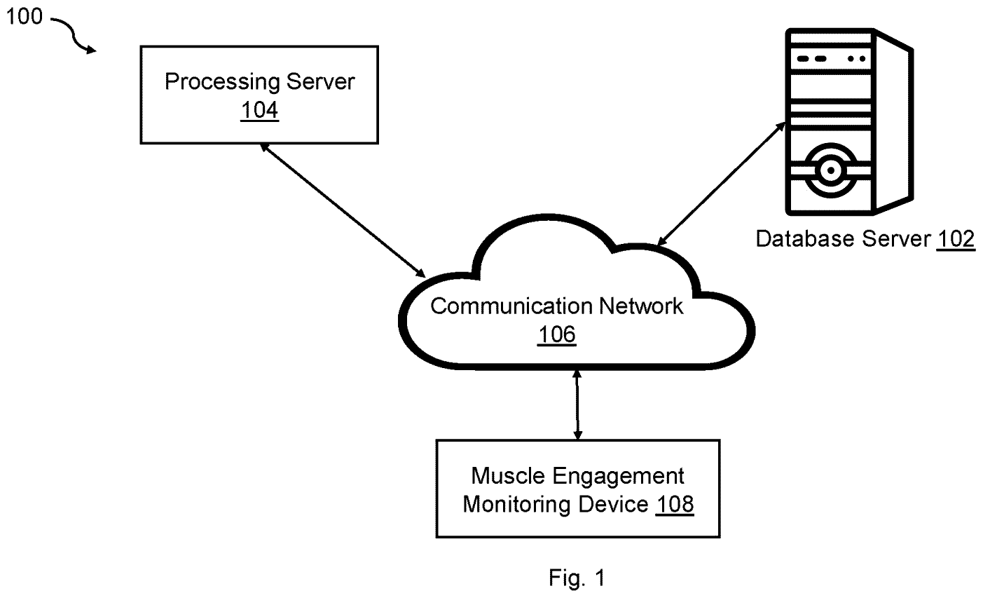

According to the present invention, FIG. 1 is a block diagram that illustrates a system environment 100 in which various embodiments of the invention may be implemented. The system environment 100 typically includes an advanced muscle engagement monitoring device 108, a processing server 104, a data storage server 102, and a communication network 106. The muscle engagement monitoring device 108 is configured to collect electromyographic data from core and lower back muscles and is communicatively linked to the processing server 104 via the communication network 106. The processing server 104, in turn, is connected to the data storage server 102, where all collected muscle activity data is securely stored and managed. These components work in unison within the system environment 100 to facilitate real-time muscle monitoring and secure communication of feedback to the user.

In some embodiments, the standalone system (without external help) may facilitate the above-discussed analysis as a requirement. For example, the user device may be capable of performing real-time monitoring and generating feedback.

FIG. 1 illustrates the system environment 100 of the present invention, showcasing how the key components namely, the data storage server 102, the processing server 104, the communication network 106, and the muscle engagement monitoring device 108 are interconnected and work together to provide real-time monitoring and feedback on core and lower back muscle engagement. The muscle engagement monitoring device 108 is worn by the user and equipped with advanced biosensors that detect electromyographic signals from the targeted muscle groups. These signals are transmitted wirelessly via the communication network 106 to the processing server 104. The processing server 104 analyzes the incoming data in real-time, generating personalized feedback and insights based on the user's muscle engagement patterns. Simultaneously, all the data-both raw and processed is securely transmitted to the data storage server 102, where it is stored for long-term analysis, retrieval, and integration with other health metrics. Such an interconnected system enables continuous, adaptive monitoring.

The muscle engagement monitoring device 108, as described in the present invention, is a wearable system specifically designed to monitor and provide real-time feedback on core and lower back muscle activity. The device is equipped with advanced biosensors that are strategically placed on the user's body to detect electromyographic signals generated by the targeted muscles during physical activities. These signals are then processed by an integrated microcontroller within the device. The device is capable of delivering immediate feedback through haptic vibrations, visual indicators, or auditory alerts, allowing users to make instant adjustments to their posture or technique. Additionally, the device communicates wirelessly with a processing server, where the data is further analyzed, stored, and integrated with other health metrics, providing a comprehensive overview of the user's muscle engagement patterns. The said real-time, adaptive feedback mechanism makes the muscle engagement monitoring device 108 an essential tool for optimizing performance.

The processing server 104, as described in the present invention, is a component that analyzes and manages the data collected by the muscle engagement monitoring device 108. Once the device captures electromyographic signals from the user's core and lower back muscles, these signals are transmitted wired or wirelessly to the processing server 104 via the communication network. The server is equipped with advanced processing capabilities which it uses to analyze the incoming data in real time. This analysis allows the server to generate personalized feedback, insights, and recommendations based on the user's unique muscle engagement patterns. Additionally, the server manages the storage of all collected data by interfacing with the data storage server 102, ensuring that the user's information is securely stored, easily accessible, and can be integrated with other health data for comprehensive monitoring and analysis. It makes the processing server 104 an integral part of the overall system, enabling intelligent data processing and continuous improvement of the user's physical performance and rehabilitation.

The data storage server 102, as described in the present invention, is a component responsible for securely storing and managing all the data collected and processed by the muscle engagement monitoring device 108 and the processing server 104. After the processing server analyzes the electromyographic data captured by the monitoring device, the processed data, along with raw signal data and user-specific insights, is transmitted to the data storage server 102. This server is designed to handle large volumes of data, ensuring that all information is stored efficiently and securely, with robust encryption protocols to protect user privacy. The data storage server 102 also facilitates easy retrieval and access to the stored data, allowing users, healthcare providers, or fitness professionals to review historical data, track progress over time, and make informed decisions based on comprehensive muscle engagement patterns. Furthermore, the server supports integration with other health monitoring systems and digital platforms, enabling a holistic view of the user's health and fitness data. By maintaining a reliable and secure repository for all collected information, the data storage server 102 ensures that the system can provide continuous, long-term support for user performance optimization.

The communication network 106, as described in the present invention, enables seamless connectivity and data exchange between the various components of the system, including the muscle engagement monitoring device 108, the processing server 104, and the data storage server 102. This network facilitates the real-time transmission of electromyographic data from the monitoring device to the processing server, where the data is analyzed using AI and machine learning modules. The communication network 106 supports wired or wireless communication protocols, such as Bluetooth, Wi-Fi, or cellular networks, ensuring that data can be transmitted quickly and reliably, even in dynamic environments. Additionally, the network allows the processed feedback and insights to be sent back to the monitoring device, enabling immediate user interaction through haptic, visual, or auditory feedback. The communication network 106 also ensures that all collected and processed data is securely transferred to the data storage server for long-term storage and analysis. By providing a robust and secure connection between all system components, the communication network 106 is essential for enabling the real-time, adaptive functionality of the system, ensuring that users receive timely and accurate feedback to optimize their physical performance based on the feedback.

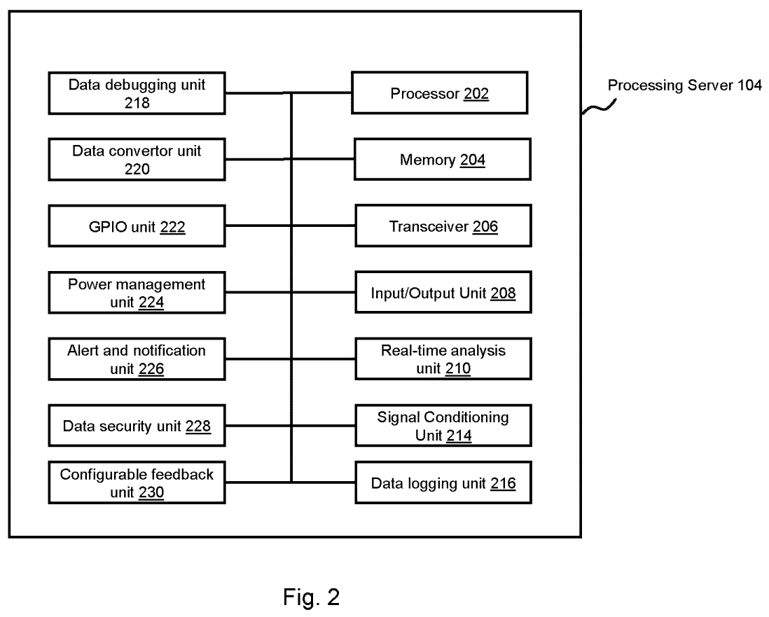

FIG. 2 is a block diagram that illustrates an operation unit configured for the system and method to provide comprehensive real-time muscle engagement monitoring and feedback, in accordance with an embodiment of the present disclosure. FIG. 2 is explained in conjunction with elements from FIG. 1. Here, the processing server 104, as part of the operation unit, preferably includes a processor 202, a memory 204, a transceiver 206, an input/output unit 208, and several specialized units for muscle data processing and management. These include a Real-Time Analysis Unit 210 for immediately assessing muscle engagement, adapting in real-time to the user's specific biomechanics and exercise routines. The Signal Conditioning Unit 214 further refines the electromyographic signals received from the monitoring device 108, ensuring that the data is clean and accurate before processing. The processor 202 collaborates with a Data Logging Unit 216 to securely store all captured and processed data. The Data Debugging Unit 218 ensures the accuracy and integrity of the data, automatically correcting any detected anomalies. Additionally, the processing server 104 features a Data Converter Unit 220 that converts analog signals to digital. The General-Purpose Input/Output (GPIO) Unit 222 allows for the integration of additional sensors or peripherals, enhancing the system's adaptability. The operation unit is equipped with a Power Management Unit 224 that oversees power supply, battery management, and configuration switching, ensuring efficient and uninterrupted operation. The system also includes an Alert and Notification Unit 226 that provides users with critical, personalized real-time feedback based on their unique muscle engagement patterns and predicted risk factors. A Data Security Unit 228 ensures the privacy and protection of user data, which is crucial for building trust in health systems. Lastly, the Configurable Feedback Unit 230 tailors the delivery of haptic, visual, or auditory outputs to each user's preferences and specific needs, making the feedback not only immediate but also highly relevant and effective.

The Processor 202, as described in the present invention, is the central component of the operation unit, responsible for managing and executing all the core functions of the system. It is equipped with advanced computational capabilities, enabling it to process the electromyographic data collected by the muscle engagement monitoring device 108 in real-time. The processor 202 analyzes this data to assess muscle engagement, adapting its feedback based on the user's specific biomechanical patterns and exercise routines.

Memory 204, as described in the present invention, is a component of the operation unit, working closely with processor 202 to store and manage all the data required for the system's advanced functionalities. This memory is designed to hold both short-term and long-term data, including real-time electromyographic signals, processed feedback, user-specific muscle engagement patterns, and historical data. The Memory 204 also stores the system's firmware and configuration settings, ensuring that the processor 202 can quickly access the necessary information to provide immediate and accurate feedback. Additionally, memory 204 plays a critical role in the continuous learning process of the system's AI components, enabling the system to adapt to the user's evolving needs over time by retaining and analyzing previous data. It allows the system to deliver increasingly personalized and effective feedback, making memory 204 an element in the system's ability to optimize performance and support long-term rehabilitation goals.

The transceiver 206, as described in the present invention, is a component of the operation unit responsible for enabling seamless wireless communication between the muscle engagement monitoring device 108, the processing server 104, and other external devices or networks. The transceiver 206 operates as both a transmitter and a receiver, facilitating the real-time exchange of electromyographic data collected by the monitoring device with the processing server 104. It ensures that processed feedback, alerts, and notifications are quickly sent back to the monitoring device, allowing users to receive immediate and relevant haptic, visual, or auditory feedback. Additionally, the transceiver 206 supports connectivity with digital health platforms and cloud services, enabling the secure transmission of user data for long-term storage, analysis, and integration with other health metrics. By maintaining a reliable and high-speed communication link, the transceiver 206 ensures that the system functions efficiently and effectively, providing users with real-time insights that enhance performance, and support their overall health and fitness goals.

The input/output unit 208, as described in the present invention, serves as an interface between the operation unit and both the user and external devices. The unit is responsible for managing all the inputs received from the muscle engagement monitoring device 108, such as electromyographic signals, user commands, and sensor data, and routing them to the appropriate processing components within the system. Simultaneously, the input/output unit 208 handles the delivery of outputs generated by the processing server 104, including real-time feedback, alerts, and notifications, back to the monitoring device or other connected interfaces. This feedback can be communicated through various modalities, such as haptic vibrations, visual displays, or auditory signals, depending on the user's preferences and the system's configuration. Additionally, the input/output unit 208 facilitates the integration of peripheral devices, such as additional sensors or external controllers, via GPIO ports, ensuring the system's adaptability to various user needs and environments. By efficiently managing both incoming data and outgoing responses, the input/output unit 208 plays a role in ensuring that the system operates smoothly, delivering timely and accurate feedback that helps users optimize their muscle engagement.

The Real-Time Analysis Unit 210, as described in the present invention, is a component within the operation unit that is responsible for the immediate processing and analysis of electromyographic data collected by the muscle engagement monitoring device 108. This unit evaluates the real-time muscle activity data, focusing specifically on core and lower back muscles. The Real-Time Analysis Unit 210 rapidly processes incoming signals, identifying patterns of muscle engagement, and determining whether the user's movements are optimal. It continuously adapts its analysis based on the user's current activity and historical data, ensuring that the feedback provided is highly relevant and personalized. The insights generated by this unit are then communicated back to the user through haptic, visual, or auditory feedback via the input/output unit 208, enabling instant corrective actions during physical activities. By providing immediate, data-driven feedback, the Real-Time Analysis Unit 210 enhances the system's ability to support effective training and rehabilitation, making it a critical component of the overall system.

The Signal Conditioning Unit 214, as described in the present invention, is a component within the operation unit responsible for preparing the raw electromyographic signals captured by the muscle engagement monitoring device 108 for accurate and reliable analysis. The unit performs essential functions such as amplifying, filtering, and normalizing the incoming signals to remove noise and interference that may distort the data. By refining these signals, the Signal Conditioning Unit 214 ensures that only clean, high-quality data is passed on to the Real-Time Analysis Unit 210 and other processing components. This precise conditioning is particularly important for detecting the subtle and specific muscle activities of the core and lower back muscles, which are crucial for the system's effectiveness in providing real-time feedback and predictive analytics. By enhancing the clarity and integrity of the electromyographic data, the Signal Conditioning Unit 214 plays a role in the system's ability to deliver accurate, actionable insights that help users optimize their performance, and achieve their fitness or rehabilitation goals.

The Data Logging Unit 216, as described in the present invention, is another component within the operation unit that is responsible for the systematic recording and storage of all electromyographic data collected by the muscle engagement monitoring device 108, as well as the processed data. This unit ensures that both raw and refined data are securely logged in real-time, creating a comprehensive historical record of the user's muscle engagement patterns, performance metrics, and feedback responses. The Data Logging Unit 216 plays a role in supporting the system's continuous learning and adaptive capabilities, as the stored data serves as a foundation for the Predictive Analytics Unit 212 to identify trends and forecast potential risks. Additionally, the data logged by this unit can be accessed for detailed post-activity analysis, long-term tracking of progress, and integration with other digital health platforms. By maintaining an accurate and complete log of all relevant data, the Data Logging Unit 216 enhances the system's ability to provide personalized, data-driven insights that evolve with the user's needs, making it a key component in optimizing performance and supporting effective rehabilitation.

The Data Debugging Unit 218, as described in the present invention, is a component within the operation unit that ensures the accuracy, integrity, and reliability of the data processed by the system. This unit is responsible for identifying, diagnosing, and correcting any anomalies or errors that may occur in the electromyographic data captured by the muscle engagement monitoring device 108 or in the subsequent data processing stages. By utilizing advanced AI modules, the Data Debugging Unit 218 continuously monitors the data flow for inconsistencies, signal distortions, or processing glitches that could impact the quality of the feedback provided to the user. When an issue is detected, this unit can automatically correct the error or, if necessary, flag the data for further review, ensuring that only high-quality, accurate information is used for real-time analysis and predictive analytics. The proactive role of the Data Debugging Unit 218 in maintaining data integrity is crucial for the system's overall performance, as it ensures that users receive reliable and precise feedback, thereby enhancing the effectiveness of the system in optimizing muscle engagement, and supporting long-term rehabilitation and fitness goals.

The Data Converter Unit 220, as described in the present invention, is a component within the operation unit responsible for transforming the raw analog signals captured by the muscle engagement monitoring device 108 into digital data that can be accurately processed by the system. This unit ensures that the electromyographic signals, which are initially captured in an analog form, are converted into precise digital representations without losing critical information or introducing noise. The Data Converter Unit 220 maintains the integrity and quality of the data as it transitions from the physical signals generated by muscle activity to the digital format required for real-time analysis, predictive analytics, and feedback generation. By providing a seamless and accurate conversion process, this unit enables the system to perform high-precision data analysis, ensuring that the feedback provided to the user is both timely and highly reliable. The Data Converter Unit 220 is essential for enabling the advanced capabilities of the system, such as personalized feedback, and performance optimization, making it a key element in the overall functionality and effectiveness of the invention.

The Input/Output (GPIO) Unit 222, as described in the present invention, is a flexible and versatile component within the operation unit that facilitates the integration of additional sensors, peripherals, and external devices with the muscle engagement monitoring system. This unit provides a range of general-purpose input/output ports that allow the system to interface with various hardware components, such as supplementary biosensors, external controllers, or other monitoring devices, expanding the system's capabilities and adaptability. The GPIO Unit 222 enables the customization and enhancement of the system by allowing users or developers to connect and configure additional inputs and outputs according to specific needs or use cases. For instance, it can be used to incorporate extra sensors for monitoring other muscle groups or to connect to external devices for advanced data visualization or control. The GPIO Unit 222 ensures that the system remains highly modular and extendable, supporting a wide array of configurations and applications, from basic muscle monitoring to complex, multi-device setups. By providing this level of connectivity and flexibility, the GPIO Unit 222 enhances the system's utility, making it suitable for a broad range of fitness, rehabilitation, and research scenarios.

The Power Management Unit 224, as described in the present invention, is a component within the operation unit responsible for ensuring efficient and reliable power distribution throughout the muscle engagement monitoring system. This unit manages the power supply to all system components, including the muscle engagement monitoring device 108, the processing server 104, and various specialized units like the Real-Time Analysis Unit 210. The Power Management Unit 224 oversees the charging and discharging cycles of the system's battery, optimizing energy use to extend operational time and ensure that the system remains functional during prolonged activities. It also includes features for power regulation, ensuring that each component receives the appropriate voltage and current to operate effectively without risking damage from power surges or drops. Additionally, the Power Management Unit 224 may incorporate intelligent power-saving modes that reduce energy consumption during periods of inactivity, further enhancing the system's efficiency. By managing power efficiently and maintaining a stable energy supply, the Power Management Unit 224 ensures that the system delivers consistent, reliable performance, enabling continuous real-time monitoring, data processing, and feedback without interruption, which is essential for optimizing user outcomes in fitness and rehabilitation.

The Alert and Notification Unit 226, as described in the present invention, is an advanced component within the operation unit that leverages artificial intelligence to deliver timely, personalized alerts and notifications to the user based on real-time data analysis and predictive insights. The unit assesses the urgency and relevance of each event, generating alerts that are specifically tailored to the user's current activity and historical patterns. These alerts can be communicated through various modalities, including haptic feedback, visual signals, or auditory notifications, ensuring that the user receives immediate and contextually appropriate guidance. For instance, if the system detects that the user is overexerting a particular muscle group, the Alert and Notification Unit 226 can promptly notify the user to adjust their form or take a break. Additionally, the unit can escalate notifications in critical situations, such as when immediate corrective action is needed, ensuring the user's safety and enhancing the effectiveness of their training or rehabilitation. This unit provides a highly responsive and intelligent layer of feedback, making the system more proactive and user-centric in its approach to optimizing muscle engagement.

The Data Security Unit 228, as described in the present invention, is a component within the operation unit dedicated to ensuring the confidentiality, integrity, and security of all data processed, stored, and transmitted by the muscle engagement monitoring system. This unit implements advanced encryption protocols and security measures to protect sensitive user information, such as electromyographic data, personal health records, and real-time feedback, from unauthorized access and potential breaches. The Data Security Unit 228 oversees secure data transmission between the monitoring device 108, the processing server 104, and the data storage server 102, using secure communication channels to prevent data interception or tampering. It also manages user authentication processes, ensuring that only authorized individuals can access the system's data and functionalities. In addition to protecting data during transmission and storage, the unit continuously monitors the system for potential security threats or vulnerabilities, automatically initiating protective actions or alerts if suspicious activity is detected. By safeguarding user data with robust security practices, the Data Security Unit 228 not only ensures compliance with privacy regulations but also builds trust in the system's use for health and fitness monitoring, making it a secure and reliable tool for users who rely on accurate and private data to optimize their performance and well-being.

The Configurable Feedback Unit 230, as described in the present invention, is an innovative component within the operation unit that provides users with tailored, real-time feedback based on their specific preferences and the system's analysis of their muscle engagement data. This unit allows for the customization of feedback delivery methods, enabling users to choose how they receive notifications and insights-whether through haptic vibrations, visual cues, auditory signals, or a combination thereof. The Configurable Feedback Unit 230 works in conjunction with the AI-enhanced Alert and Notification Unit 226 to ensure that feedback is not only immediate and relevant but also presented in a manner that is most effective and comfortable for the user. For instance, during a high-intensity workout, the system might prioritize haptic feedback to allow the user to maintain focus without needing to look at a screen, while during a cool-down phase, visual or auditory feedback might be more appropriate. The unit's adaptability also extends to adjusting the intensity, frequency, and type of feedback based on the user's activity, muscle engagement patterns, and personal settings. This level of customization ensures that the feedback is aligned with the user's individual needs and preferences, enhancing the system's usability and effectiveness in optimizing muscle performance, and supporting rehabilitation efforts. The Configurable Feedback Unit 230 is a key feature that makes the system versatile and user-friendly, offering a highly personalized experience that maximizes the benefits of real-time muscle monitoring.

In an exemplary operation, a system to monitor and provide real-time feedback on core and lower back muscle engagement during physical activities is disclosed. The system comprises advanced biosensors that detect electromyographic signals from the targeted muscles, a processing server integrated within the operation unit that analyzes these signals, and a communication network that facilitates the seamless exchange of data between the components. The system also includes a data storage server for securely managing and storing both raw and processed data. In an embodiment, the system features a Real-Time Analysis Unit that provides immediate assessments of muscle engagement, enabling users to make on-the-fly adjustments to optimize performance. Additionally, the system incorporates a Configurable Feedback Unit, which allows users to customize how they receive feedback-whether through haptic, visual, or auditory signals ensuring that the feedback is tailored to their specific needs and preferences. In yet another embodiment, the system includes a Data Security Unit that protects user data with advanced encryption and security measures, ensuring confidentiality and integrity across all operations.

In an embodiment, the processor is configured to analyze real-time electromyographic data from the biosensors, using AI and machine learning modules to provide immediate feedback on muscle engagement. In another embodiment, the processor is configured to adapt its analysis based on the user's historical data, continuously learning and refining its feedback to better match the user's unique muscle patterns and activity levels. In yet another embodiment, the processor is configured to manage predictive analytics. Additionally, the processor is configured to interface with the Configurable Feedback Unit, allowing it to deliver personalized feedback through haptic, visual, or auditory signals based on the user's preferences. Furthermore, the processor is configured to ensure data integrity by working with the Data Debugging Unit to detect and correct any anomalies in the data, thereby maintaining the accuracy and reliability of the system's outputs.

In another embodiment of the present invention, the system is designed to integrate seamlessly with external digital health platforms and wearable devices. This embodiment allows the system to pull in additional data such as heart rate, sleep patterns, and physical activity levels from connected devices, which the processor then incorporates into its analysis to provide even more personalized and context-aware feedback. The system can also synchronize with mobile apps, enabling users to track their muscle engagement progress over time, set personalized goals, and receive detailed reports that combine muscle data with other health metrics. This holistic approach not only enhances the accuracy and relevance of the feedback provided during workouts but also supports long-term health and wellness by helping users make informed decisions based on a broad spectrum of physiological data. Additionally, this embodiment includes advanced data-sharing features, allowing users to securely share their progress and insights with healthcare providers, trainers, or physical therapists for more tailored guidance and support.

In yet another embodiment of the present invention, the system is equipped with an adaptive training mode that adjusts the intensity and type of feedback based on the user's current physical condition and performance goals. The processor dynamically assesses the user's real-time muscle engagement, fatigue levels, and overall workout intensity, using this data to modulate the feedback provided by the system. For instance, during a high-intensity workout, the system may increase the frequency and intensity of haptic feedback to ensure the user maintains proper form, while during recovery phases, it might shift to more subtle visual or auditory cues to encourage gentle muscle engagement without overexertion. This adaptive training mode can also be programmed to align with specific training regimens, such as strength building, endurance training, or rehabilitation, offering customized feedback that evolves with the user's progress. This embodiment enhances the system's ability to support a wide range of fitness goals, providing a more responsive and individualized training experience that helps users achieve their desired outcomes safely and effectively.

Let us consider a practical scenario to illustrate the workings of the present disclosure. Consider an individual recovering from a lower back injury who is using the system to ensure proper muscle engagement during physical therapy exercises. The individual wears the muscle engagement monitoring device, which is equipped with biosensors placed on the core and lower back muscles. As the individual performs specific rehabilitation exercises, the system's processor analyzes real-time electromyographic data, ensuring that the targeted muscles are engaged correctly and at the appropriate intensity. If the system detects improper form or muscle overexertion, the Alert and Notification Unit immediately provides haptic feedback, prompting the individual to adjust their posture or reduce intensity. Throughout the session, data is logged and stored securely, allowing the physical therapist to review the patient's progress and adjust the therapy plan as needed. This scenario demonstrates how the system supports safe, effective rehabilitation by providing real-time, personalized feedback that guides the individual through their recovery process.

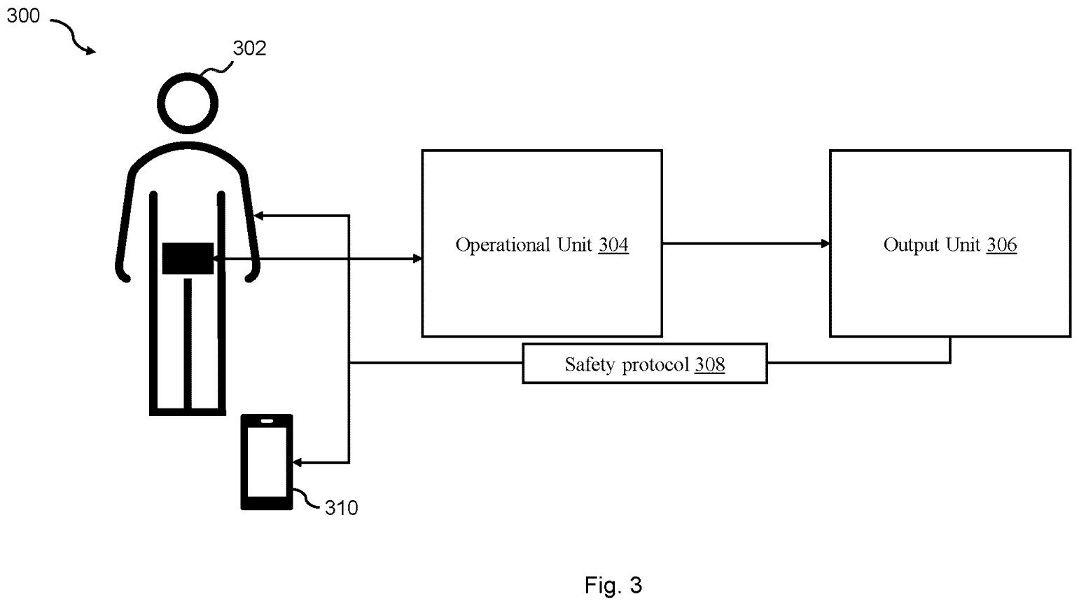

FIG. 3 illustrates a system 300 for monitoring and providing real-time feedback on muscle engagement, in accordance with an embodiment of the present disclosure. System 300 includes a bio unit 302, an operational unit 304, an output unit 306, and a safety protocol 306. The components of the System 300 may be coupled with each other through a wireless or wired communication medium.

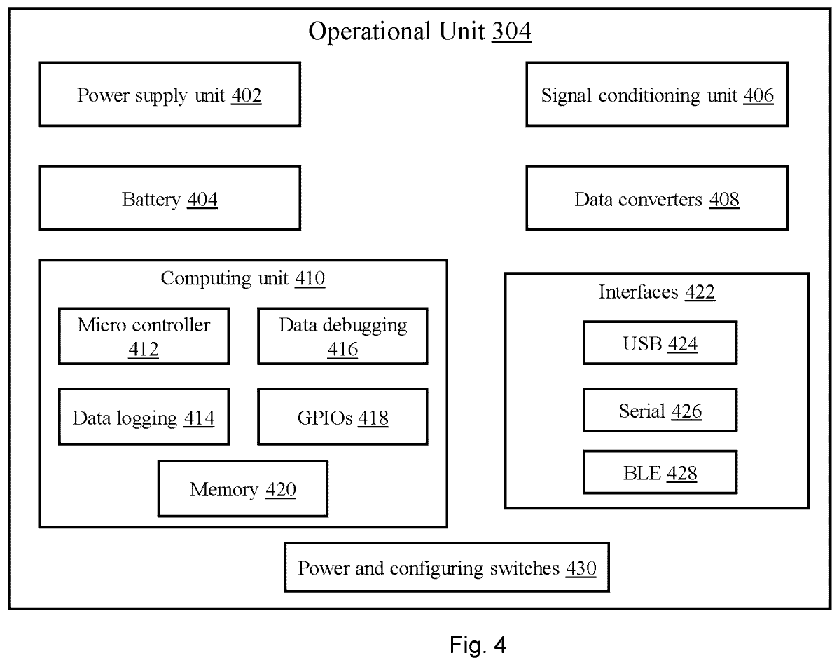

Bio unit 302 includes a sensing device physically coupled with the user. In an implementation, the sensing unit may comprise multiple biosensors, such as an encoder, a load cell, a torque sensor, an electromyogram (EMG) sensor, a proprioceptor, a force-sensitive resistor (FSR), etc. The bio unit 302 detects electromyographic signals from the targeted muscles and transmits the electromyographic signals to the operational unit 304. The Operational Unit 304 includes multiple components for processing the electromyographic signals. Such components of the operational unit 304 are described in detail through FIG. 4.

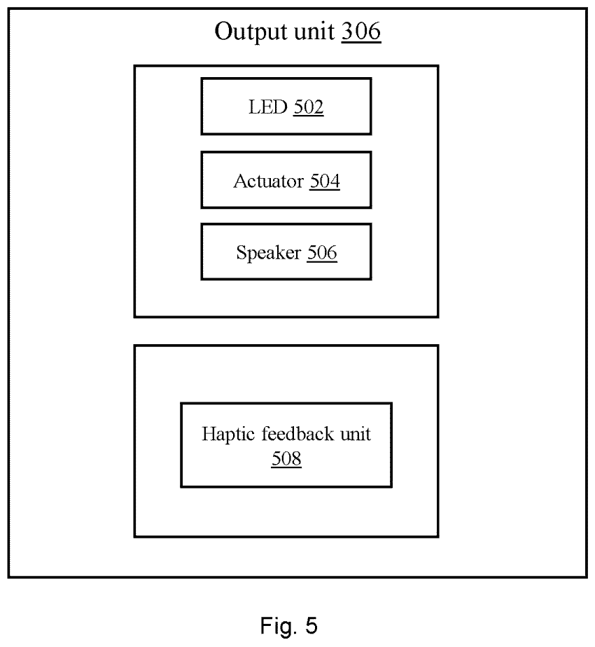

The operational unit 304 transmits the processed data to the output unit 306. The output unit 306 includes multiple components for notifying the user. Such components of the output unit 306 are described in detail in FIG. 5. The processed data are securely stored in a memory for future reference and analysis. The data is transmitted to the user through a safety protocol 308.

In some embodiments, the data may be transmitted to a user device 310, such as a mobile phone, a Personal Computer (PC), or a datalogger gadget. The user device 310 is connected to the system using a wired or wireless medium. The user device 310 is equipped with an application for providing an external communication capability to the system.

FIG. 4 illustrates a block diagram of the operational unit 304, in accordance with an embodiment of the present disclosure. The operational unit 304 includes a power supply unit 402, a battery 404, a signal conditioning unit 406, a data converter 408, a computing unit 410, interfaces 422, and power and configuration switches 430.

The power supply unit 402 is configured to supply power to various components of the operational unit 304 through the battery 404. The signal conditioning unit 406 is configured to filter noise from the electromyographic signals received from the bio unit 302. The data converter 408 is configured to convert the electromyographic signals to a format understandable by the computing unit 410.

The computing unit 410 is configured to process the converted data to generate activity data. The computing unit 410 includes a microcontroller (alternately referred to as a processor) 412, a data logger 141, a data debugger 416, a GPIO 418, and a memory 420. The microcontroller 412 is configured to perform all the calculations, such as analyzing the obtained electromyographic signals in real time to determine the muscle activity of the user. The muscle activity is indicative of movement of core and/or lower back muscles. Further, the microcontroller 412 generates activity data based on the muscle activity of the user. The micro-controller 412 is further configured to securely store raw data and processed data in memory for future reference and analysis using the data logger 414. The micro-controller 412 is further configured to protect the integrity and confidentiality of the data processed, stored, and transmitted using an encryption method using the data debugger 416. The micro-controller 412 is further configured to obtain the user's historical data from the memory 420 using the GPIO 418. The microcontroller 412 determines individual muscle engagement patterns based on the user's historical data. The micro-controller 412 further continuously updates the feedback based on the individual muscle engagement patterns. The microcontroller 412 is further configured to allow the user to customize the type, intensity, and mode of feedback delivery according to their preferences.

The operational unit 304 transmits and receives the data using the interface 422. The interface 422 includes a USB 424, a serial bus 426, and a BLE 428 for performing communication with the bio unit 302.

FIG. 5 illustrates a block diagram of the output unit 306, in accordance with an embodiment of the present disclosure. The output unit 306 includes a Light Emitting Diode (LED) 502, an actuator 504, a speaker 506, and a haptic feedback unit 508.

The LED 502 is configured to produce a light effect in response to alert data produced by the output unit 306. The light effect may be indicative of the alert to the user. The actuator 504 is configured to generate movement of the muscles of the user in response to the alert data produced by the output unit 306. The Speaker 506 is configured to produce sound effects in response to the alert data produced by the output unit 306.

The haptic feedback unit 508 is configured to produce a haptic effect in response to the alert data produced by the output unit 306. For example, the haptic feedback unit 508 may produce a vibration in response to the alert data produced by the output unit 306.

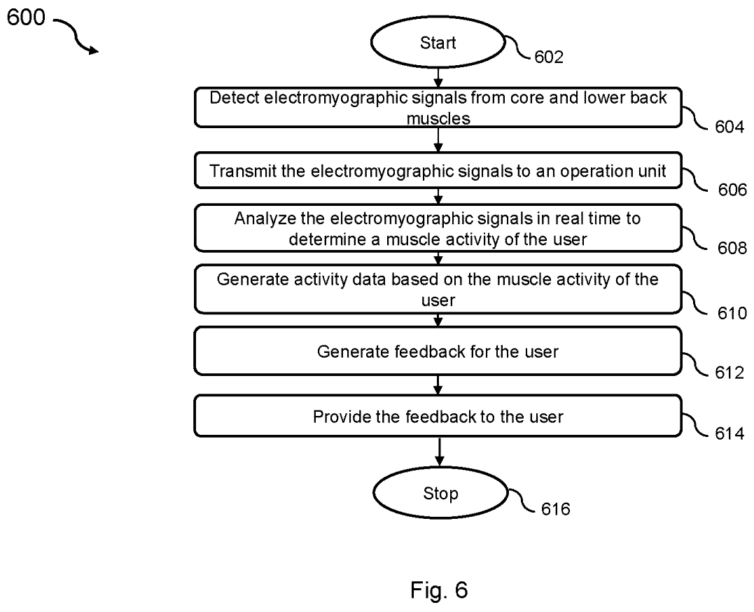

FIG. 6 illustrates a flowchart of method 600 for monitoring and providing real-time feedback on muscle engagement, in accordance with an embodiment of the present disclosure. The method begins in a Start step 602 and proceeds to step 604. At step 604, the system may detect electromyographic signals from the core and lower back muscles using a sensing unit. The sensing unit comprises a plurality of biosensors, such as an encoder, a load cell, a torque sensor, an electromyogram (EMG) sensor, a proprioceptor, a force-sensitive resistor (FSR), etc. At step 606, the system may transmit the electromyographic signals to the operational unit. The operational unit may comprise a processor for processing the signals. At step 608, the system may analyze the electromyographic signals in real time to determine the muscle activity of the user. The muscle activity is indicative of movement of core and/or lower back muscles. At step 610, the system may generate activity data based on the muscle activity of the user. The activity data is provided to an output unit. At step 612, the system may generate feedback for the user based on the activity data. At step 614, the system may provide feedback to the user through one or more of a haptic output, a visual output, and an auditory output.

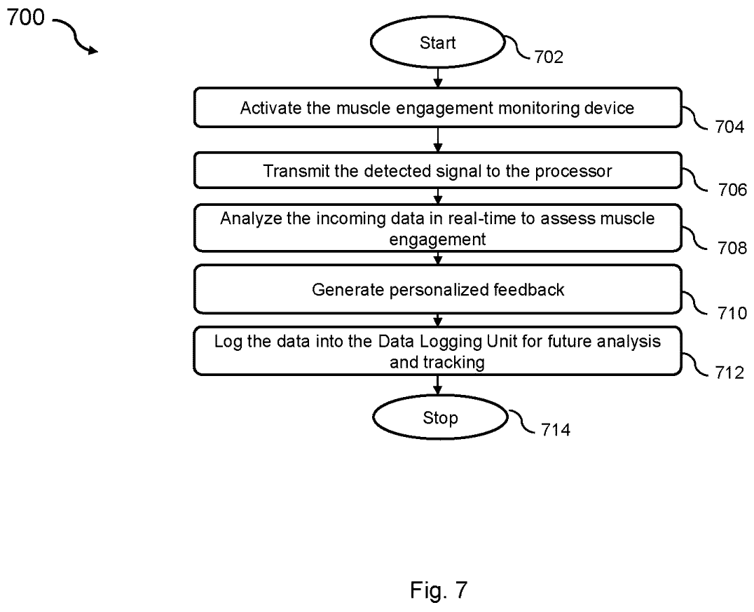

FIG. 7 is a flowchart that illustrates method 700 for providing real-time feedback on core and lower back muscle engagement, in accordance with an embodiment of the present disclosure. The method 700 begins in a Start step 702 and proceeds to a step 704. At step 704, the system activates the muscle engagement monitoring device, initializing the biosensors to start detecting electromyographic signals from the user's core and lower back muscles. The method then moves to step 706, where the detected signals are transmitted via the communication network to the processor within the operation unit. At step 708, the processor sends the signals to an external server, equipped with AI and machine learning modules, and analyzes the incoming data in real-time to assess muscle engagement. In step 710, based on the analysis, the system generates personalized feedback through the Configurable Feedback Unit, delivering it to the user via haptic, visual, or auditory outputs. At step 712, the system logs the data into the Data Logging Unit for future analysis and tracking. Finally, it proceeds to an End step 716 where the session data is securely stored, and the system powers down or enters a standby mode until the next use.

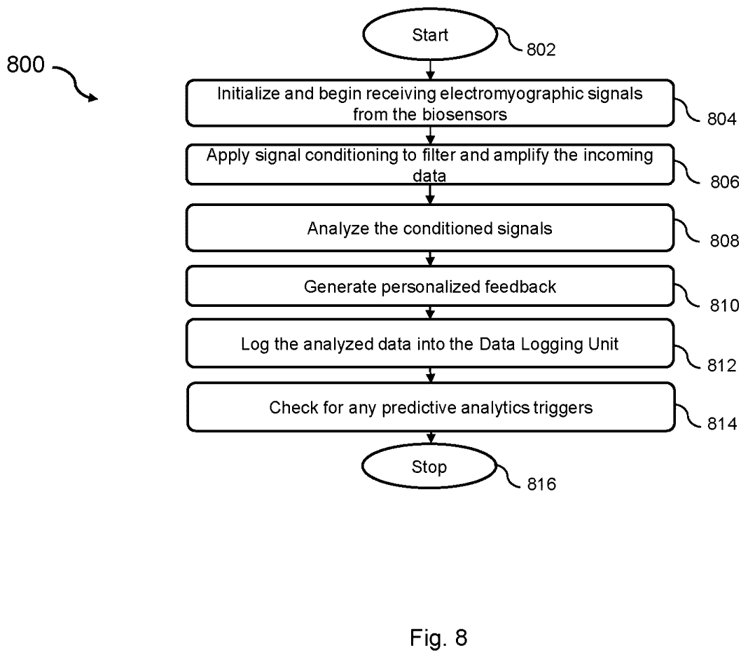

FIG. 8 is a flowchart that illustrates how the processor is configured to operate within the system to monitor and provide real-time feedback on core and lower back muscle engagement, in accordance with an embodiment of the present invention. The method begins in a Start step 802 and proceeds to step 804. At step 804, the processor initializes and begins receiving electromyographic signals from the biosensors attached to the user's core and lower back muscles. Moving to step 806, the processor applies signal conditioning to filter and amplify the incoming data, ensuring that the electromyographic signals are clean and ready for precise analysis.

At step 808, the processor sends the data to an external server that uses AI and machine learning modules to analyze the conditioned signals, determining the level of muscle engagement and identifying any anomalies or signs of improper form. In step 810, based on this analysis, the processor generates personalized feedback, which is then routed to the Configurable Feedback Unit for immediate delivery to the user through haptic, visual, or auditory means.

The method continues to step 812, where the processor logs the analyzed data into the Data Logging Unit, ensuring that all relevant information is stored for future reference and ongoing machine learning enhancements. At step 814, the processor checks for any predictive analytics triggers. If such conditions are detected, the processor adjusts the feedback accordingly and may issue proactive alerts to the user. Finally, the method proceeds to an End step 818, where the processor finalizes data storage, securely transmits any necessary information to connected health platforms, and powers down or enters a low-power standby mode until the next session.

The present disclosure provides a concrete and tangible solution to a significant technical problem in the field of muscle engagement monitoring and real-time feedback during physical activities. Specifically, it addresses the challenges of accurately monitoring core and lower back muscles. The present disclosure offers specific technical features and functionalities, such as advanced biosensors that precisely detect electromyographic signals from targeted muscle groups and a processor equipped with AI and machine learning modules that analyze these signals in real-time. Additionally, the system includes a Configurable Feedback Unit that delivers personalized feedback through haptic, visual, or auditory means, ensuring that users receive the most relevant and effective guidance during their activities. Furthermore, the integration of a Data Logging Unit for comprehensive data tracking and a Data Security Unit for safeguarding user information ensures that the system is both robust and secure, offering a comprehensive and reliable solution for enhancing muscle performance and supporting rehabilitation efforts.

The present disclosure offers several technical advantages over conventional muscle monitoring systems, making it a superior solution for real-time feedback. Firstly, its integration of advanced biosensors specifically designed to detect electromyographic signals from core and lower back muscles provides a level of precision and specificity that conventional systems lack. This enhanced accuracy ensures that users receive highly relevant feedback, directly targeting the muscle groups critical for stability and performance. Additionally, the use of AI and machine learning modules within the processor enables the system to analyze data in real-time, adapt to individual user patterns, and continuously improve the accuracy of its feedback. This dynamic and personalized approach is a significant advancement over static, one-size-fits-all solutions. The inclusion of a Configurable Feedback Unit further enhances the user experience by allowing for tailored feedback through haptic, visual, or auditory signals, making the system adaptable to various user preferences and activity types. The Data Logging Unit offers comprehensive tracking and historical analysis, supporting long-term improvement and rehabilitation, while the Data Security Unit ensures that all user data is protected with robust encryption and secure communication protocols. Together, these technical advancements make the present disclosure a highly effective, user-friendly, and secure system for optimizing muscle engagement.

The present disclosure may be realized in hardware, or a combination of hardware and software. The present disclosure may be realized in a centralized fashion, in at least one computer system, or in a distributed fashion, where different elements may be spread across several interconnected computer systems. A computer system or other apparatus adapted for carrying out the methods described herein may be suited. A combination of hardware and software may be a general-purpose computer system with a computer program that, when loaded and executed, may control the computer system such that it carries out the methods described herein. The present disclosure may be realized in hardware that comprises a portion of an integrated circuit that also performs other functions.

A person with ordinary skills in the art will appreciate that the systems, modules, and sub-modules have been illustrated and explained to serve as examples and should not be considered limiting in any manner. It will be further appreciated that the variants of the above-disclosed system elements, modules, and other features and functions, or alternatives thereof, may be combined to create other different systems or applications. Those skilled in the art will appreciate that any of the aforementioned steps and/or system modules may be suitably replaced, reordered, or removed, and additional steps and/or system modules may be inserted, depending on the needs of a particular application. In addition, the systems of the aforementioned embodiments may be implemented using a wide variety of suitable processes and system modules, and are not limited to any particular computer hardware, software, middleware, firmware, microcode, and the like. The claims can encompass embodiments for hardware and software or a combination thereof. While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

We claim:

1. A system for monitoring and providing real-time feedback on muscle engagement, comprising:
a sensing unit comprising a plurality of biosensors physically coupled on a user, wherein the plurality of biosensors is configured to detect electromyographic signals from the core and lower back muscles of the user;
an operational unit communicatively coupled with the plurality of biosensors, wherein the operation unit comprises a processor configured to:
obtain the electromyographic signals from the operational unit;
analyze the obtained electromyographic signals in real-time to determine a muscle activity of the user, wherein the muscle activity is indicative of movement of core and/or lower back muscles; and
generate activity data based on the muscle activity of the user; and
an output unit communicatively coupled with the operational unit, wherein the output unit is configured to:
generate feedback for the user, based on the activity data; and
provide the feedback to the user through one or more of a haptic output, a visual output, and an auditory output.

2. The system of claim 1, wherein the processor is further configured to securely store raw data and processed data in a memory for future reference and analysis.

3. The system of claim 1, wherein the processor is further configured to protect the integrity and confidentiality of the data processed, stored, and transmitted using an encryption method.

4. The system of claim 1, wherein the processor is further configured to:
obtain user's historical data from the memory;
determine, using the Machine Learning (ML) model, individual muscle engagement patterns based on the user's historical data; and
continuously update the feedback based on the individual muscle engagement patterns.

5. The system of claim 1, wherein the processor is further configured to allow the user to customize the type, intensity, and mode of feedback delivery according to their preferences.

6. The system of claim 1, further comprising a communication network configured to facilitate the transmission of electromyographic data between the sensing unit, the operational unit, and the output unit.

7. The system of claim 1, further comprising a power management unit configured to manage power distribution, battery charging, and energy efficiency across all components of the system.

8. A method for monitoring and providing real-time feedback on muscle engagement, comprising:
detecting electromyographic signals from core and lower back muscles using a sensing unit that comprises a plurality of biosensors;
transmitting the electromyographic signals to an operation unit comprising a processor;
analyzing, by the processor, the electromyographic signals in real-time to determine a muscle activity of the user, wherein the muscle activity is indicative of movement of core and/or lower back muscles;
generating, by the processor, activity data based on the muscle activity of the user, wherein the activity data is provided to an output unit;
generating, by the output unit, feedback for the user based on the activity data; and
providing, by the output unit, the feedback to the user through one or more of a haptic output, a visual output, and an auditory output.

9. The method of claim 8, further comprising securely storing raw data and processed data in a memory for future reference and analysis.

10. The method of claim 8, further comprising protecting the integrity and confidentiality of the data processed, stored, and transmitted using an encryption method.

11. The method of claim 8, further comprising:
obtaining user's historical data from the memory;
determining, using the Machine Learning (ML) model, individual muscle engagement patterns based on user's historical data; and
continuously updating the feedback based on the individual muscle engagement patterns.

12. The method of claim 8, further comprising allowing the user to customize the type, intensity, and mode of feedback delivery according to their preferences.
